(19) United States
(12) Patent Application Publication
(10) Pub. No.: US 2026/0248980 A1
(43) Pub. Date: Aug. 27, 2026

(54) AIR DECONTAMINATION SYSTEM

(71) Applicant: THE UNIVERSITY OF LIVERPOOL, Liverpool (GB)

(72) Inventors: James WALSH, Liverpool (GB); Aaron DICKENSON, Liverpool (GB); Mohammad HASAN, Liverpool (GB)

(21) Appl. No.: 18/874,638

(22) PCT Filed: Jun. 14, 2023
(86) PCT No.: PCT/GB2023/051554

§ 371 Date: Dec. 13, 2024

(30) Foreign Application Priority Data
Jun. 17, 2022 (GB) 2208962.7

(51) Int. Cl. A61L 9/22

(52) U.S. Cl. CPC A61L 9/22; A61L 2209/11

(57) ABSTRACT

There is provided an air decontamination system (100). The air decontamination system (100) comprises a conduit for air (110), a pulse generator (130) and a plurality of plasma generation devices (120). The pulse generator (130) is couplable to a power supply and configured to generate a first pulse having a first pulse width of a first predetermined time, thereby controlling airflow between the plurality of plasma generation devices (120) in the conduit (110). The plurality of plasma generation devices (120) is located on two interior surfaces (111, 112) of the conduit (110). Each plasma generation device (120) comprises a high voltage electrode (121) and a ground electrode (122), the electrodes (121, 122) coupled to the pulse generator (130), and a dielectric barrier (123) between the high voltage electrode (121) and the ground electrode (122). The electrodes (121, 122) are configured to apply a voltage across the dielectric barrier (123) to generate a plasma on a surface of the dielectric barrier (123), thereby decontaminating surrounding air.

### TECHNICAL FIELD

The invention relates to an air decontamination system and a method of decontaminating air by an air decontamination system.

### BACKGROUND

Airborne contaminants encompass various pathogens (e.g., bacteria and viruses), allergens (e.g., pollen, animal hair and spores) and pollutants (e.g., cleaning chemicals, fuels). Each of these contaminants can negatively impact human health. Therefore, there is a need to reduce the presence of airborne contaminants in homes, vehicles, workspaces and similar.

A number of types of existing air decontamination systems are known. For instance, systems comprising a filter to remove airborne contaminants are known. However, such systems do not kill micro-organisms, meaning that contaminants may accumulate and grow on the filter. Many existing air decontamination systems include moving parts for directing airflow. Disadvantageously, moving parts, such as fans, are prone to malfunction and/or blockage and are noisy.

Hence, there is a need for an air decontamination system that does not rely on filtration or moving parts. Overall, there is a desire to provide an efficient, consumable-fee, low-cost air decontamination system that facilitates safe eradication of airborne contaminants.

### SUMMARY

It is one aim of the invention, amongst others, to provide an airborne decontamination system which at least partially obviates or mitigates at least some of the disadvantages of the prior art, whether identified herein or elsewhere, or to provide an alternative approach. For instance, it is an aim of embodiments of the invention to provide an air decontamination system that facilitates safe eradication of airborne contaminants.

According to the invention there is provided an air decontamination system and a method of decontaminating air by an air decontamination system. Other features of the invention will be apparent from the dependent claims, and the description that follows.

According to a first aspect of the invention, there is provided an air decontamination system. The air decontamination system comprises a conduit for air, a pulse generator and a plurality of plasma generation devices. The pulse generator is couplable to a power supply and configured to generate a first pulse having a first pulse width of a first predetermined time, thereby controlling airflow between a plurality of plasma generation devices in the conduit. The plurality of plasma generation devices is located on two interior surfaces of the conduit. Each plasma generation device comprises a high voltage electrode and a ground electrode, the electrodes coupled to the pulse generator, and a dielectric barrier between the high voltage electrode and the ground electrode. The electrodes are configured to apply a voltage across the dielectric barrier to generate a plasma on a surface of the dielectric barrier, thereby decontaminating surrounding air.

The air decontamination system may further comprise a controller configured to control operation of the plurality of plasma generation devices.

The controller may be configured to cause the plurality of air decontamination devices to operate asynchronously.

The first predetermined time may be greater than 20 μs, whereby plasma generation device operation is in a forward flow mode.

The pulse generator may be configured to generate a second pulse having a second pulse width of a second predetermined time.

The second predetermined time may be less than 20 μs, whereby plasma generation device operation is in a reverse flow mode.

The pulse generator may be configured to generate a third pulse of a third predetermined time, whereby plasma generation device operation is in a split flow mode.

The controller may be configured to switch the plurality of plasma generation devices between at least two of: the forward flow mode, the reverse flow mode and the split flow mode.

The pulse generator may be configured to generate pulses having continually varying pulse widths, thereby directing air 0° to 180° relative to the surface of the dielectric barrier.

The pulse generator may be configured to generate pulses having a frequency of 5 kHz to 25 kHz.

The air decontamination system may further comprise an aperture through the dielectric barrier through which air is directed.

The power supply may be configured to provide a voltage of 2 kV to 12 kV.

The dielectric barrier may have a dielectric constant of 3.1 to 10.

The dielectric barrier may have a thickness of is 0.25 mm to 3 mm.

According to a second aspect of the invention, there is provided a method of decontaminating air by an air decontamination system comprising: a conduit for air; a pulse generator couplable to a power supply; and a plurality of plasma generation devices located on two interior surfaces of the conduit, each comprising a high voltage electrode and a ground electrode, the electrodes coupled to the pulse generator, and a dielectric barrier between the high voltage electrode and the ground electrode. The method comprises applying a voltage across the dielectric barrier to generate a plasma on a surface of the dielectric barrier, thereby decontaminating surrounding air; and controlling airflow between the plurality of plasma generation devices in the conduit by generating a first pulse having a first pulse width of a first predetermined time.

### BRIEF DESCRIPTION OF THE DRAWINGS

For a better understanding of the invention, and to show how exemplary embodiments of the same may be brought into effect, reference will be made, by way of example only, to the accompanying diagrammatic Figures, in which:

FIG. 1 schematically depicts an air decontamination system according to an exemplary embodiment;

FIG. 2 schematically depicts electrodes of a plasma generation device according to an exemplary embodiment;

FIG. 3 schematically depicts a velocity profile of a plasma generation device for four different pulse widths according to an exemplary embodiment;

FIG. 4 schematically depicts the relationship between airflow direction and pulse width according to an exemplary embodiment;

FIG. 5 schematically depicts airflow produced in a conduit by the plurality of plasma generation devices according to an exemplary embodiment; and

FIG. 6 schematically depicts a method of decontaminating air using the air decontamination system shown in FIG. 1 according to an exemplary embodiment.

### DETAILED DESCRIPTION

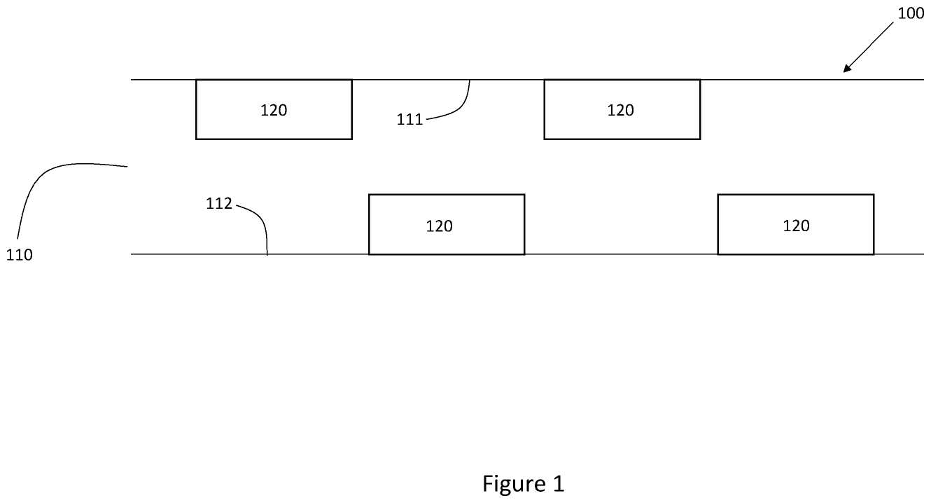

FIG. 1 schematically depicts an air decontamination system 100 according to an exemplary embodiment. The air decontamination system 100 comprises a conduit for air 110, typically in the form of a channel or tube, a pulse generator (not shown) and a plurality of plasma generation devices 120.

The conduit 110 is not limited to a particular length, and the length of the conduit 110 may be adapted according to the dimensions of the site where the air decontamination system 100 is to be installed. Similarly, there is no requirement for a particular number or spacing of the plasma generation devices 120, though more closely spaced plasma generation devices may enhance certain effects described below.

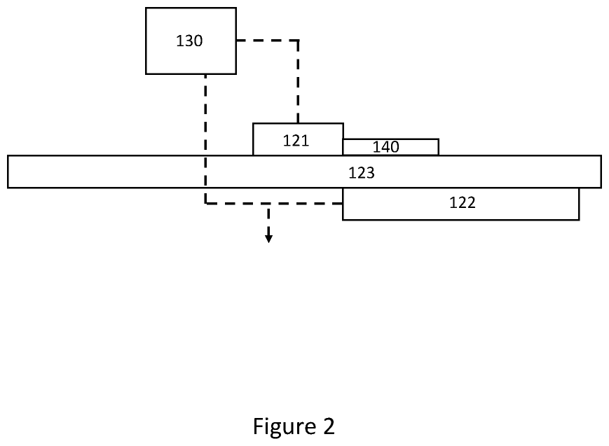

As shown in FIG. 2, each of the plurality of plasma generation devices 120 comprises a high voltage electrode 121 and a ground electrode 122. The electrodes 121, 122 are coupled to the pulse generator 130. Copper is a suitable material for the electrodes, but other known suitable material(s) may be used.

A dielectric barrier 123 is located between the high voltage electrode 121 and the ground electrode 122. Typically, the dielectric barrier 123 has a dielectric constant of 3.1 to 10 and a thickness of is 0.25 mm to 3 mm. Preferably, the dielectric barrier 123 comprises quartz. The dielectric barrier 123 may form, at least in part, the conduit 110. Depending on the dielectric material, one of the electrodes 122, 123 could be placed on the outside of the conduit 110 and the other of the electrodes 122, 123 on the inside conduit.

The electrodes 121, 122 are configured to apply a voltage across the dielectric barrier 123 to generate a plasma on a surface of the dielectric barrier 123. In generating the plasma, surrounding air molecules are ionised and are accelerated through or between the electrodes 121, 122, the air being decontaminated in the process. Decontaminating the air means completely or partially eradicating contaminants (e.g., killing all or some bacteria, destroying some or all allergens/pollutants). The box 140 in FIG. 2 indicates the plasma region resulting from application of a voltage across the dielectric barrier 123.

Advantageously, no moving parts are required by the air decontamination system 100, such moving parts being susceptible to malfunction and/or blockages. Relatedly, the absence of any form of pump or fan means that the air decontamination system 100 does not produce significant noise. Further, there is neither a physical medium nor consumables that could be blocked, increasing the reliability of the decontamination system 100 compared with types of known decontamination systems.

The pulse generator 130 may be integrated with one or more (e.g., each) of the plurality of plasma generation devices 120 or may be a discrete unit of the decontamination system 100. The pulse generator 130 is couplable to a power supply, which is typically configured to provide a voltage of 2 kV to 12 kV. The pulse generator 130 is typically configured to generate pulses having a frequency of 5 kHz to 25 kHz.

It has been found that by generating the plasma using pulse widths in certain ranges, air can be drawn in and ejected at particular angles. In this way, airflow between plasma generation devices 120 in the conduit 110 can be controlled. In other words, air can be steered through the conduit 110. By steering air through the conduit 110, the efficacy air decontamination system 100 is increased in comparison with using a single plasma generation device 120. For instance, by directing the air from one plasma generation device 120 to the next, the contact time (i.e., the time that the air is exposed to the plasma) is increased compared with using a single plasma generation device 120.

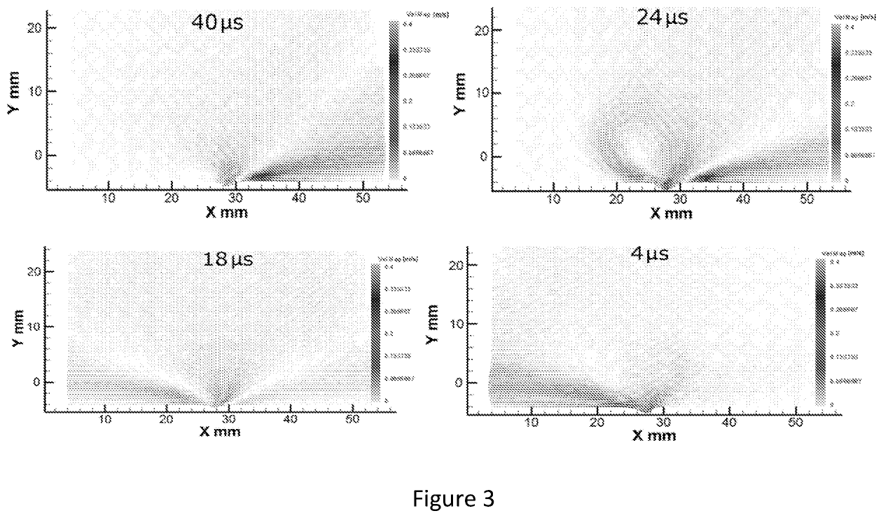

FIG. 3 schematically depicts a velocity profile of a plasma generation device for four different pulse widths according to an exemplary embodiment, illustrating drawing in air and ejecting it at particular angles. Comparing a velocity profile measured at 40 μs (top left graph) with a velocity profile measured at 4 μs (bottom right graph) shows a marked change in the direction of airflow.

As shown in FIG. 1, the plurality of plasma generation devices 120 is located on two interior surfaces 111, 112 of the conduit 110. As shown in FIG. 1, the plurality of plasma generation devices is typically located alternately (e.g., in a zig-zag arrangement) on each of the interior surfaces 111, 112 along the length of the conduit 110. This arrangement of the plurality of plasma generation devices 120 facilitates effective decontamination by increasing contact time compared with an arrangement in which all the plasma generation devices 120 are located on the same interior surface 111, 112 of the conduit 110.

The pulse generator 130 is configured to generate a first pulse having a first pulse width of a first predetermined time. In one example, the first predetermined time is greater than 20 μs. It has been found that a pulse width greater than 20 μs causes air to be directed in a direction parallel to the dielectric medium. In the case of a pulse width greater than 20 μs, one or more of the plurality of plasma generation devices 120 to which this pulse width is applied are said to operate in a forward flow mode.

The pulse generator 130 may be configured to generate a second pulse having a second pulse width of a second predetermined time. In one example, the second predetermined time is less than 20 μs. It has been found that a pulse width less than 20 μs causes air to be directed in a direction parallel to the dielectric medium 123 but opposite to the direction when the pulse width is greater than 20 μs. In the case of a pulse width less than 20 μs, one or more of the plurality of plasma generation devices 120 to which this pulse width is applied are said to operate in a reverse flow mode. Advantageously, the direction of airflow can be changed by changing pulse width—there is no need to alter the geometry of the air decontamination system 100 to change the direction of airflow. In contrast, known technologies using sinusoidal waves only permit airflow in one direction.

The pulse generator 130 may be configured to generate a third pulse of a third predetermined time. In one example, the third predetermined time is 20 μs. It has been found that a pulse width of 20 μs causes the airflow to be split between the forward flow mode and the reverse flow mode (split mode). Relatedly, the pulse generator 130, which may be a unit comprising a plurality of pulse generators, may be configured to generate pulses having continually varying pulse widths, thereby directing air 0° to 180° relative to the surface of the dielectric barrier 123. Advantageously, a split flow or directing air 0° to 180° relative to the surface of the dielectric barrier 123 facilitates vortex airflow in the conduit 110. In other words, a split flow or directing air 0° to 180° relative to the surface of the dielectric barrier 123 facilitates turbulence as opposed to laminar airflow. Advantageously, vortex airflow further prolongs contact time between contaminated air and plasma.

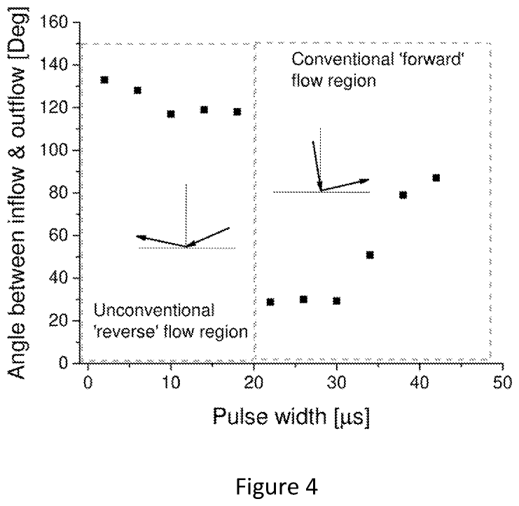

FIG. 4 schematically depicts the relationship between airflow direction and pulse width according to an exemplary embodiment. FIG. 4 shows the angle between the airflow into a plasma generation device 120 and the airflow out of that plasma generation device 120 in degrees over a range of pulse widths. As mentioned, at a pulse width of below 20 μs the angular relationship changes compared with a pulse width of greater than 20 μs such that the airflow changes from what is denoted a forward flow to a reverse flow. The forward flow and the reverse flow correspond to the respective regimes shown in the right side and left side of the graph in FIG. 4.

The air decontamination system 100 may further comprise a controller configured to control operation of the plurality of plasma generation devices 120. For instance, the controller may be configured to cause the plurality of air decontamination devices 120 to operate asynchronously. For instance, the controller may be configured to switch the plurality of plasma generation devices 120 between at least two of: the forward flow mode, the reverse flow mode and the split flow mode. Advantageously, asynchronous operation of the plasma generation devices 120 facilitates vortex airflow in the conduit 110, which further prolongs contact time.

There may be an aperture through the dielectric barrier 123 through which air is directed. The aperture may be a rectangular or circular. For example, the electrodes 121, 122 may be in the form of strips or rings. Advantageously, ring-shaped electrodes may be configured for blowing/sucking (i.e., air flow is directed through the ring electrodes).

The below table summarises appropriate ranges for each of the aforementioned parameters, as well as specifying their respective relationships (far right column).

| Parameter | Range | Relation to Other Parameters |
|---|---|---|
| Voltage | 2 kV to 12 kV | The voltage required to create a uniform |
| amplitude | | plasma, which is a function of all other |
| | | parameters in addition to the smoothness of |
| | | the electrodes and the dielectric. |
| Repetition | 5 kHz to 25 kHz | The lower end of repetition frequency |
| frequency | | increases the pulse width window where the |
| | | flow is reversed (stays within the parameter |
| | | range given below). |
| Pulse | 150 ns to 100 μs | The lower end of the pulse width requires |
| width | | voltage amplitude closer to the higher end of |
| | | the range (maximum available pulse width |
| | | depends on frequency) |
| Dielectric | 3.1 to 10 | The higher end of the dielectric constant |
| constant | | requires voltage amplitude closer to the lower |
| | | end of the range. |
| Dielectric | 0.25 mm to 3 mm | The lower end of the dielectric thickness |
| thickness | | requires voltage amplitude closer to the lower |
| | | end of the range. |

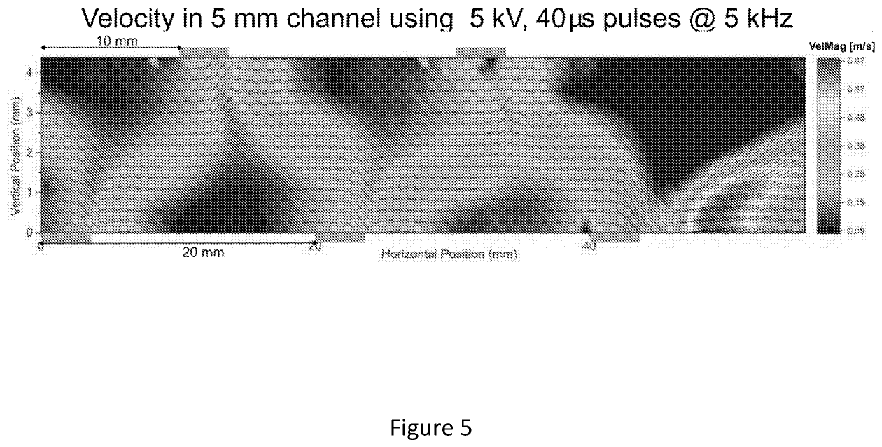

FIG. 5 schematically depicts airflow produced in a conduit by the plurality of plasma generation devices 120 according to an exemplary embodiment. The arrangement of the plurality of plasma generation devices 120 corresponds to that shown in FIG. 1. The conduit 110 is a channel having a width of 4.5 mm. The plasma generation devices 120 are spaced apart by 20 mm on each one of the interior surfaces 111, 112 of the conduit 110. The voltage amplitude and the repetition frequency are 5 kV and 5 kHz, respectively. The pulse width is 40 μs, which corresponds to operation of the plurality of plasma generation devices 120 in a forward flow mode. As discussed, this arrangement of the plurality of plasma generation devices 120 facilitates effective decontamination by increasing contact time compared with an arrangement in which all the plasma generation devices 120 are located on the same interior surface 111, 112 of the conduit 110.

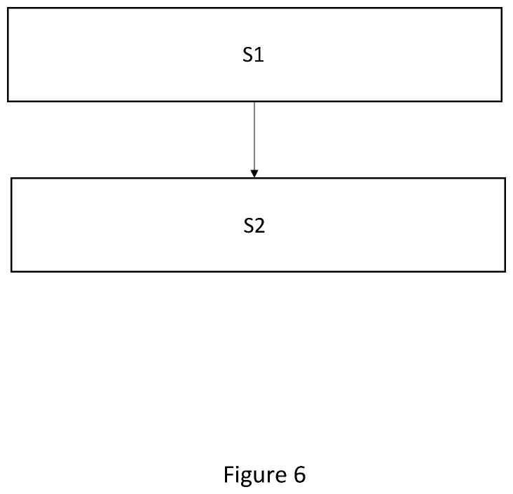

As shown in FIG. 6, a method of decontaminating air by the air decontamination system 100 applying S1 a voltage across the dielectric barrier 123 to generate a plasma on a surface of the dielectric barrier 123, thereby decontaminating surrounding air; and controlling S2 airflow between the plurality of plasma generation devices 120 in the conduit by generating a first pulse having a first pulse width of a first predetermined time.

The method shown in FIG. 6 may further comprise controlling the plasma generation devices 120 to operate synchronously, to operate in a forward flow mode, to operate in a reverse flow mode or to operate in a split flow mode, as well as to switch between these modes, as described above.

In summary, the invention provides an air decontamination system 100 and a method of using the decontamination system 100 that do not rely on moving parts, resulting in reliability and an absence of excess noise, that decontaminate the air and that have improved efficacy due to increasing contact time for decontamination.

Although a preferred embodiment has been shown and described, it will be appreciated by those skilled in the art that various changes and modifications might be made without departing from the scope of the invention, as defined in the appended claims and as described above.

The optional features set out herein may be used either individually or in combination with each other where appropriate and particularly in the combinations as set out in the accompanying claims. The optional features for each aspect or exemplary embodiment of the invention, as set out herein are also applicable to all other aspects or exemplary embodiments of the invention, where appropriate. In other words, the skilled person reading this specification should consider the optional features for each aspect or exemplary embodiment of the invention as interchangeable and combinable between different aspects and exemplary embodiments.

All of the features disclosed in this specification (including any accompanying claims and drawings), and/or all of the steps of any method or process so disclosed, may be combined in any combination, except combinations where at most some of such features and/or steps are mutually exclusive.

Each feature disclosed in this specification (including any accompanying claims, and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims and drawings), or to any novel one, or any novel combination, of the steps of any method or process so disclosed.

1. An air decontamination system comprising:
a conduit for air; and
a pulse generator couplable to a power supply and configured to generate a first pulse having a first pulse width of a first predetermined time, thereby controlling airflow between a plurality of plasma generation devices in the conduit;
wherein the plurality of plasma generation devices is located on two interior surfaces of the conduit, each plasma generation device comprising:
a high voltage electrode and a ground electrode, the electrodes coupled to the pulse generator; and
a dielectric barrier between the high voltage electrode and the ground electrode, wherein the electrodes are configured to apply a voltage across the dielectric barrier to generate a plasma on a surface of the dielectric barrier, thereby decontaminating surrounding air.

2. The air decontamination system according claim 1, further comprising:
a controller configured to control operation of the plurality of plasma generation devices.

3. The air decontamination system according to claim 23, wherein the controller is configured to cause the plurality of air decontamination devices to operate asynchronously.

4. The air decontamination system according to claim 1, wherein the first predetermined time is greater than 20 μs, whereby plasma generation device operation is in a forward flow mode.

5. The air decontamination system according to claim 1, wherein the pulse generator is configured to generate a second pulse having a second pulse width of a second predetermined time.

6. The air decontamination system according to claim 5, wherein the second predetermined time is less than 20 μs, whereby plasma generation device operation is in a reverse flow mode.

7. The air decontamination system according to claim 1, wherein the pulse generator is configured to generate a third pulse of a third predetermined time, whereby plasma generation device operation is in a split flow mode.

8. The air decontamination system according to claim 7, wherein the controller is configured to switch the plurality of plasma generation devices between at least two of: the forward flow mode, the reverse flow mode and the split flow mode.

9. The air decontamination system according to claim 1, wherein the pulse generator is configured to generate pulses having continually varying pulse widths, thereby directing air 0° to 180° relative to the surface of the dielectric barrier.

10. The air decontamination system according to claim 1, wherein the pulse generator is configured to generate pulses having a frequency of 5 kHz to 25 kHz.

11. The air decontamination system according to claim 1, further comprising:
an aperture through the dielectric barrier through which air is directed.

12. The air decontamination system according to claim 1, wherein the power supply is configured to provide a voltage of 2 kV to 12 kV.

13. The air decontamination system according to claim 1, wherein the dielectric barrier has a dielectric constant of 3.1 to 10.

14. The air decontamination system according to claim 1, wherein the dielectric barrier has a thickness of is 0.25 mm to 3 mm.

15. A method of decontaminating air by an air decontamination system comprising: a conduit for air; a pulse generator couplable to a power supply; and a plurality of plasma generation devices located on two interior surfaces of the conduit, each comprising a high voltage electrode and a ground electrode, the electrodes coupled to the pulse generator, and a dielectric barrier between the high voltage electrode and the ground electrode, the method comprising:
applying a voltage across the dielectric barrier to generate a plasma on a surface of the dielectric barrier, thereby decontaminating surrounding air; and
controlling airflow between the plurality of plasma generation devices in the conduit by generating a first pulse having a first pulse width of a first predetermined time.
